(19) United States
(12) Patent Application Publication
(10) Pub. No.: US 2009/0062423 A1
(43) Pub. Date: Mar. 5, 2009

(54) ORTHOPAEDIC CEMENT MIXTURES WITH LOW WEIGHT PERCENT POLYVINYL ALCOHOL (PVA) SOLUTION

(75) Inventors: Randal R. Betz, Ocean City, NJ (US); Rebeccah Brown, Decatur, GA (US); Stephen Williams, Montpelier, VT (US)

Correspondence Address: MYERS BIGEL SIBLEY & SAJOVEC, PO BOX 37428, RALEIGH, NC, 27627, US

(73) Assignees: Salumedica, LLC, Atlanta, GA (US); Spinemedica Corporation, Marietta, GA (US)

(21) Appl. No.: 12/194,980

(22) Filed: Aug. 20, 2008

Related U.S. Application Data
(60) Provisional application No. 60/968,709, filed on Aug. 29, 2007.

(51) Int. Cl. C08L 29/04; A61B 17/58

(52) U.S. Cl. 523/116; 606/93

(57) ABSTRACT

Orthopaedic bone cement mixtures include a primary material that has a flowable state and a solid mass state and a low weight percent amount of polyvinyl alcohol (PVA) material. The PVA material is distributed in the primary material.

### RELATED APPLICATION

This application claims the benefit of priority of U.S. Provisional Application Ser. No. 60/968,709 filed Aug. 29, 2007, the contents of which are hereby incorporated by reference as if recited in full herein.

### FIELD OF THE INVENTION

The invention relates to bone void or fracture filler material and may be particularly suitable for treating osteoporosis and/or collapsed or fractured vertebral bones or other cancellous bone areas such as calcneus and proximal tibia.

### BACKGROUND OF THE INVENTION

Vertebroplasty is an image-guided, minimally invasive, non-surgical therapy used to strengthen or stabilize a broken vertebra that has been weakened by disease or in response to a therapy, such as osteoporosis or cancer. Vertebroplasty is accomplished by injecting an orthopedic cement mixture into a fissure, void or bone fracture. Vertebroplasty is commonly used to treat pain caused by osteoporotic compression fractures.

Kyphoplasty is also a minimally invasive (spinal) surgical procedure. Kyphoplasty is often used to treat patients that suffer from spinal stenosis or herniated discs. In this procedure, a balloon is used to create a cavity and restore a collapsed vertebral bone back to near normal height. Once the desired cavity size is created by the balloon, the balloon is typically removed and cement is flowably introduced into the cavity. The cement then hardens relatively quickly to maintain the reconstruction height.

### SUMMARY OF EMBODIMENTS OF THE INVENTION

Embodiments of the invention are directed to biocompatible bone cement and/or bone void filler mixtures comprising a low weight percent polyvinyl alcohol (PVA) solution.

Some embodiments are directed to orthopaedic bone cement mixtures that include a primary material that has a flowable state and a solid mass state, and a low weight percent amount of polyvinyl alcohol (PVA) material (typically in a solution). The PVA material is distributed in the primary material providing flexibility to the cement.

The PVA material may remain uncured as the primary material hardens to a solid mass in situ. The mixture may self-harden at body temperature. The low weight percent of PVA may be between about 1% to about 10%. The mixture can be viscous and flowable at certain temperatures, typically temperatures above about 80° F., typically between at least 80-85° F., and, in some embodiments, can harden to the solid mass in less than about 60 minutes when exposed to temperatures at about 98.6° F.

Some embodiments are directed to orthopaedic medical kits that include a sterile container of flowable bone cement comprising a low weight percent of PVA solution, the PVA having a molecular weight between about 124,000 to about 165,000.

The container may define a therapeutic delivery device for placing the flowable bone cement in local target bone structure in a desired quantity. Alternatively, the container may communicate or cooperate with a separate therapeutic delivery device for placing the flowable bone cement in local target bone structure in a desired quantity.

Other embodiments are directed to orthopaedic medical kits that include: (a) a sterile container of a quantity of flowable bone cement; and (b) a sterile container of PVA or PVA solution, the PVA having a molecular weight between about 124,000 to about 165,000. The PVA is provided in a quantity that when mixed with a solution and a quantity of flowable bone cement forms a low weight percent of the solution and the cement mixture.

Still other embodiments are directed to preformed bone implants that include a low weight percent of PVA. The PVA has a molecular weight of between about 124,000 to about 165,000. The PVA material is distributed throughout the implant, typically by means of a suspension or solution combined with the cement.

In particular embodiments, the implant can be a dental implant or filler for a tooth.

Some embodiments are directed to methods of embedding a bone cement at a bony site. The methods include: (a) providing a flowable viscous mixture comprising a primary material and a low weight percent amount of polyvinyl alcohol (PVA) material (e.g., crystals, pellets, or low percent weight PVA solution); and (b) introducing the flowable viscous mixture to a target bony treatment site whereby the flowable viscous mixture hardens to a solid mass.

The target boney treatment site may be an intevertebral disc. Yet other embodiments are directed to methods of fabricating biocompatible bone cements. The methods include: (a) providing a primary material or mixture of materials defining a flowable bone cement mixture that hardens to a solid mass when placed in situ; and (b) mixing a low weight percent amount of PVA material (e.g., low percentage weight PVA solution) with the bone cement. When combined with the flowable bone cement mixture, the PVA material does not substantially change the viscosity of the flowable bone cement mixture and does not substantially change (extend or shorten) the time to harden the bone cement in situ.

The PVA material can be configured so as to not chemically react with the bone cement mixture as it hardens and/or is not cured nor polymerized.

Further features, advantages and details of the present invention will be appreciated by those of ordinary skill in the art from a reading of the figures and the detailed description of the embodiments that follow, such description being merely illustrative of the present invention.

### BRIEF DESCRIPTION OF THE DRAWINGS

FIG. 1 is a schematic illustration of operations that can be used to carry out embodiments of the present invention.

FIG. 2A is a schematic illustration of a flowable orthopaedic cement with low weight percent PVA being placed into an intervertebral disc according to embodiments of the present invention.

FIG. 2B is a schematic illustration of the flowable cement placed in FIG. 2A solidified in situ to a solid mass with increased flexibility over the cement without the PVA.

FIG. 3 is a schematic illustration of the flowable orthopaedic cement with low weight percent PVA being placed into an intervertebral disc to repair a fissure, bone void or fracture according to embodiments of the present invention.

FIG. 4A is a schematic illustration of the orthopaedic cement used to affix an implantable prosthesis to local bone structure according to embodiments of the invention.

FIG. 4B is a schematic illustration of the orthopaedic cement used to repair or reinforce non-spinal bones according to yet other embodiments of the present invention.

FIG. 5A is a schematic illustration of a medical kit with the cement pre-mixed with the low weight percent PVA and packaged in a delivery device according to embodiments of the present invention.

FIG. 5B is a schematic illustration of a medical kit with the cement pre-mixed with the low weight percent PVA and packaged in a collapsible “squeeze” package with a known and labeled shelf life according to embodiments of the invention.

FIG. 5C is a schematic illustration of a medical kit with a primary cement material and the PVA material and/or solution held in separate packages for in situ mixing just prior or during delivery to the target site according to yet other embodiments of the present invention.

FIG. 5D is a schematic illustration of a medical kit with primary cement materials, PVA and liquid all in separate containers according to embodiments of the present invention.

FIG. 5E is a schematic illustration of a medical kit with a heater and delivery device according to embodiments of the present invention.

FIG. 6 is a schematic illustration of a dental application of bone cement with low weight percent PVA according to yet other embodiments of the present invention.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The term “bone cement” means a flowable viscous material that can harden into a solid mass that can do at least one of the following: (a) fill bone voids or fissures; and (b) (adhesively) attach to and/or structurally engage local tissue (e.g., bone) structure. The bone cement is a mixture of a primary material(s) and low weight percent PVA. The term “low weight percent PVA” refers to a bone cement mixture that has an amount of PVA that is between about 0.05% to about 20%, typically between about 1% to about 15%, and in particular embodiments between about 1% to about 10%. Embodiments of the invention may be particularly suitable for human and/or veterinarian use.

The primary materials forming the bone cement can be resorbable or non-resorbable, while the PVA will be non-resorbable. Examples of bone cement materials include, but are not limited to one or more of the following, calcium phosphates, including one or more of crystalline apatitic calcium phosphates and tricalcium phosphates, and/or calcium sulfates. See, e.g., U.S. Pat. Nos. 5,276,070; 6,027,742; 6,632,235; 6,428,576; 6,437,018; 6,479,565; 6,911,212; 6,949,251; 6,953,594; 7,018,460; 7,019,192; 7,094,286; and U.S. Patent Application Publication No. US 2007/0048382. The contents of these documents are hereby incorporated by reference as if recited in full herein.

The term “flexible” means that the solid (bone cement) mass can elastically flex, side-to-side and/or top to bottom, or at another angle, at least to a small degree, in response to mechanical loads placed thereon. Typically, the solid mass formed by the solidified or hardened bone cement has limited compressibility but can elastically deform under high tensile, torsion or compressive loading to inhibit fracture or splintering thereof in situ after engaged to local bone structure such that the solid mass substantially functions as normal bone. The solid mass formed by the PVA cement can have a lower compressive modulus, lower hardness, be less brittle than natural bone or conventional bone cement, and can be viscoelastic.

The term “bone cement” refers to the solid mass of bone cement combined with the low weight percent PVA. In some embodiments, the bone cement can be pre-formed and hardened into an implant configuration at a fabrication facility. The PVA can be mixed with a solution at an application site (e.g., Operating Room “OR”) or premixed and held separate from the cement mixture. In some embodiments, a low percentage weight amount of PVA crystals, pellets, powder and/or granules can be mixed with water or saline or other liquid, and heated to dissolve the PVA into a suspension or solution. The PVA solution can be packaged and supplied as a suspension or solution to be mixed with the other cement material. The PVA/liquid mixture or solution may be heated slightly to get it to flow better when combining with the other cement mixtures and/or when flowably introducing the PVA cement mixture to a target surgical site. The other cement materials can be supplied in two or three components, and can be mixed together in the OR. In other embodiments, (dry) PVA can be supplied for subsequent mixing with the cement material in the OR (operating room) or other clinical facility or may be packaged in a pre-mixed formulation. However, increased flexibility may be obtained by mixing a (very) low weight percent of PVA and liquid, then combining this with the cement material(s).

Typically, the PVA material is non-reactive with the primary material of the bone cement and is distributed through the mixture both in the flowable form and the solid mass thereby providing flexibility to the cement. That is, while the primary material may comprise a reactive composition of two or more materials, the PVA material is not cured, polymerized or chemically reacted to the primary material, before, during or after the mixture transitions to the solid mass. The “free” PVA material can be captured inside the solid mass body and/or by the local bone structure so as to inhibit systemic migration away from the target site. The bone cement with the low weight percent PVA can be self-hardening in situ in the body or in vitro. The bone cement mixture with low weight percent PVA (solution) can be flowable at temperatures above about 80° F., and may also be flowable at room temperatures, such as at temperatures between about 50° F. (10° C.) and 86° F. (30° C.). The bone cement with the low weight percent PVA may also be flowable above 50° F. (10° C.) and below about 86° F. (30° C.), and can self-harden, in situ, to the solid mass at about 98.6° F. (37° C.). However, in other embodiments, additional heat can be applied locally in vivo to the bone cement PVA mixture to cause the mixture to have less viscosity or increased flowablility and/or to cause the mixture to change from a flowable state to harden to the solid mass. In some formulations of the bone cement with the PVA, other means may be employed to cause the flowable state to transform to the solid mass, such as, for example, a chemical reaction with a reactive agent, and/or application of a curing light (via an optic probe) or energy source, such as ultraviolet light, RF energy, X-ray or photon energy and the like.

Flowability of the PVA cement mixture can depend on the content/composition of the bone cement mixture and the percent PVA. Shelf life with cement (unmixed) and PVA solution kept separately may be at least one year, and typically may be between 2-3 years at room temperature. Once mixed, a much shorter shelf-life may exist, and the combined PVA cement mixture may have a use time from the mixing of less than about 1 hour, e.g., a one-hour shelf life at room temperature. Refrigeration after production and prior to use may be appropriate to help extend the shelf life.

Some embodiments of the bone cement material and low weight percent of PVA may include a first primary material and a biocompatible tertiary material, such as, for example, collagen, hyaluron, protein, other polymer and/or other natural or synthetic materials that are configured to have a desired range of elastomeric mechanical properties.

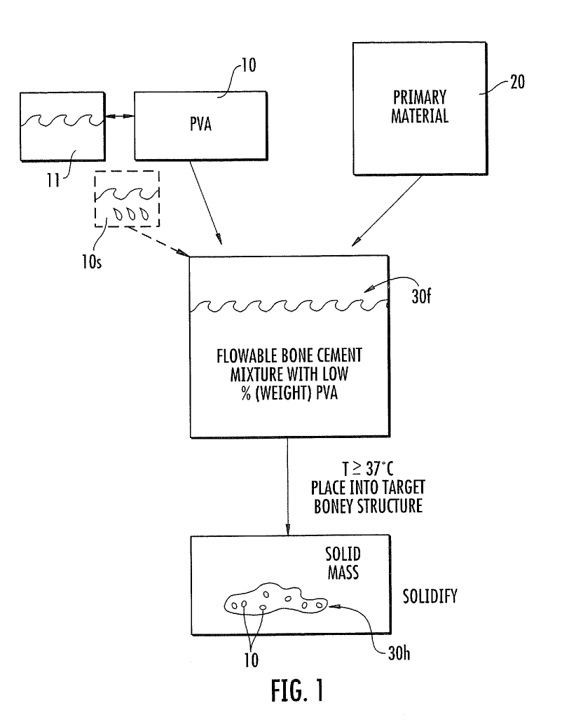

Referring to FIG. 1, PVA material 10 and primary bone cement material 20 can be mixed together to form a flowable bone cement mixture 30f. The flowable bone cement mixture 30f is configured to be applied to bony structure and harden into a solid mass 30h with distributed PVA material 10.

The primary material 20 and/or the PVA material 10 may each respectively include a solvent or liquid component. The PVA material 10 can be in any desired form, such as in a liquid solution or dry. The PVA material 10 can be held in powder, crystal, granule and/or pellet form and a sterile liquid can be packaged separate from the PVA material for onsite mixture. Alternatively, the PVA material 10 can be combined with a liquid 11 to form a low weight percent PVA solution 10s that is subsequently combined with the primary material 20. Heat can be applied to increase the solubility of the PVA material 10 in the solution 10s. The PVA material 10 can be supplied as one component of a kit with sterile liquid 11, that a clinician or user can combine at a use site or the PVA 10 can be pre-mixed with the liquid 11 and packaged as a medical grade low weight percent PVA solution 10s.

Alternatively, the primary material 20 and the PVA material 10 may be added together in a solid dry formulation, then the solvent and/or liquid added thereto to form the mixture 30.

In some embodiments, the PVA material 10 can be added to the mixture 30 in two different forms, such as, for example, powder and pellets or with some part of the PVA solubilized in liquid 11 (e.g., sterile saline) and some dry. The PVA material 10 can have a molecular weight of between about 124,000 to about 165,000. An example of a suitable PVA material, according to embodiments of the invention, is a PVA biomaterial available from SaluMedica, Inc., located in Atlanta, Ga. In particular embodiments, a low weight percent PVA solution comprises calcium sulfate-hemihydrate that forms a flexible cement.

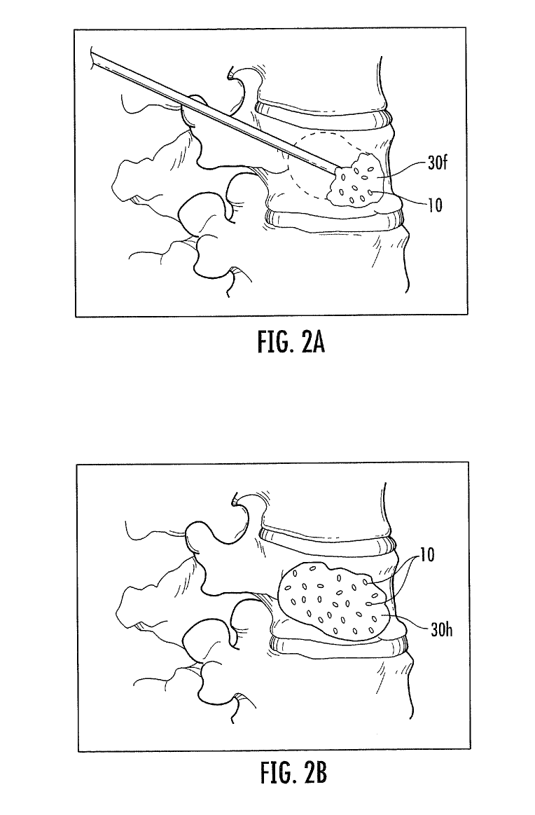

FIG. 2A illustrates the mixture 30f being placed in an intevertebral bone during a kyphoplasty procedure. FIG. 2B illustrates the hardened solid mass in position and attached to local bony structure with the distributed PVA. FIG. 3 illustrates the bone cement 30h as used for a vertebroplasty procedure.

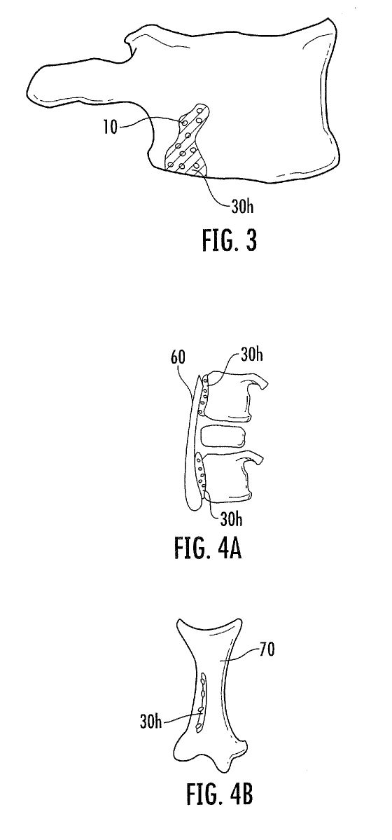

FIG. 4A illustrates that the bone cement 30h can be used to affix an implant to local bony structure. In the embodiment shown, the implant is a fusion plate 60. The cement 30h can be used with other implants or fixation members as well to stabilize, reinforce or promote attachment relatively quickly.

FIG. 4B illustrates that the bone cement 30h can be used as a bone void filler or stabilizer in non-spinal bones 70 (shown as an arm or leg bone). The bone cement 30h can be used in fissures or as a surface or bone stabilizer or reinforcements such as to strengthen the intramedullary canal for accepting an implant.

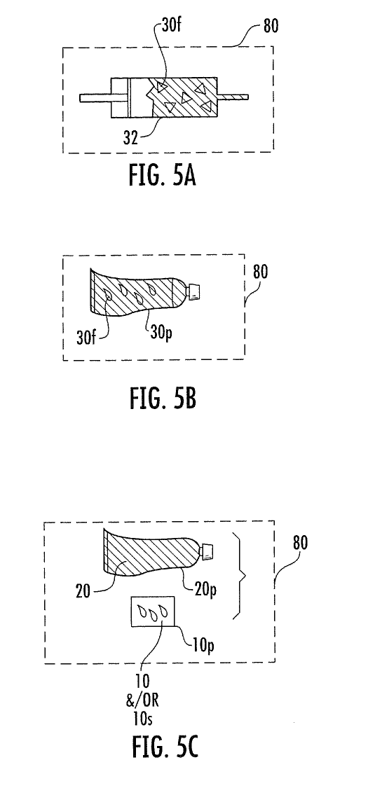

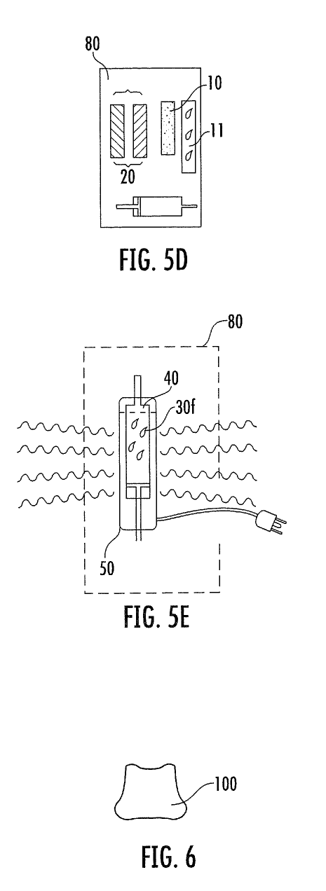

FIG. 5A is a schematic illustration of a medical kit 80 with the mixture 30f in a delivery device 32 (shown as a syringe) that can directly inject or place the mixture 30f in the target space or may connect to tubing, a needle or other delivery probe.

FIG. 5B illustrates the medical kit 80 can include the mixture 30f in a collapsible flexible sterile package 30p. In FIGS. 5A and 5B, the PVA material 10 is pre-mixed with the primary bone cement material 20 and stored in a sterile package for subsequent clinical use.

Alternatively, as shown in the kit 80 in FIG. 5C, the PVA material 10 or solution 10s can be held in a separate package 10p from the primary bone cement material 20 and package 20p and mixed just prior to or during placement in the body. The bone cement material 20 may include several materials in the package 20p that are also combined onsite, typically before adding the PVA 10 or 10s.

As shown in FIG. 5D, the kit 80 can include at least two separate containers of primary cement material 20, a container with PVA 10, and a container with liquid 11. The kit 80 can optionally include a mixing container and/or delivery device such as a syringe 50 and mixing instructions (not shown) to create the flexible cement.

As shown in FIG. 5E, the kit 80 may include a disposable or reusable portable heater 50 that is sized to hold a delivery device, such as a syringe 50, and heat the PVA 10, 10s and/or combined flowable cement and PVA 30f prior to insertion into a target surgical location. The heater 50 can include an electrical connector or include a battery for self-powering and suitable temperature limitations and/or a timer (not shown).

Table I below provides examples of different types and/or amounts of dry and wet kit contents that may be provided in different amounts for different size repairs.

TABLE 1
Exemplary Kit Contents
Example Kit Contents

| | | Component | Sm | | Md | | Lg | |
|---|---|---|---|---|---|---|---|---|
| KIT A | Dry | Cement solids | 20 | g | 50 | g | 100 | g |
| | Wet | PVA Solution | 4 | cc | 10 | cc | 20 | cc |
| KIT B | Dry | Cement solids | 20 | g | 50 | g | 100 | g |
| | | PVA | 0.2 | g | 0.5 | g | 1.0 | g |
| | Wet | Saline or other | 4 | cc | 10 | cc | 20 | cc |
| | | liquid | | | | | | |
| Potential | Dry | Cement solids X | | | | | | |
| Components | | Solid Y | | | | | | |
| | | PVA | | | | | | |
| | Wet | PVA Solution | | | | | | |
| | | Saline | | | | | | |
| | | Liquid Z | | | | | | |

In addition, the PVA solutions (percentage PVA) can be formulated to substantially match or accommodate a patient's elastic modulus of the target bone structure. Alternatively, the kits can be formulated and packaged with different low weight percents of PVA solution so as to provide different elastic modulus ranges and labeled with the particular range of elastic modulus for matching to a particular site and/or patient. In some embodiments, the elastic modulus of the low weight percent PVA bone cement can correspond to the local anatomical structure. Typically, the material can have a human bone compressive elastic modulus between about 0.01 to about 20 GPa. In particular embodiments, the implanted material can have a modulus of elasticity in tension of between about 10-20 GPa and in compression of between about 3-6 GPa. For equine, canine, feline or other veterinarian uses, the values may vary. For example, equine can have a modulus of elasticity in tension of between about 16-26 GPa, and a modulus of elasticity in compression of between about 7-10 GPa.

FIG. 6 illustrates one example of a dental bone device 100 formed of bone cement with low weight percent PVA. The mixture 30f can be placed in a mold and hardened to the solid mass to form the implant 100 or inserted into a target void, fracture or space in a natural or synthetic tooth.

The bone cement may be suitable for repair, reinforcement, stabilization, and/or fixation or attachment purposes, in, for example bone fractures, osteoporosis, dental uses or implants, other load or non-load bearing structures and/or implants, and the like.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed:

1. An orthopaedic bone cement mixture, comprising:
a primary material that has a flowable state and a solid mass state; and
a low weight percent amount of polyvinyl alcohol (PVA) material in a liquid solution, wherein the PVA material is distributed in the primary material.

2. A mixture according to claim 1, wherein the PVA material remains uncured as the primary material hardens to a solid mass in situ.

3. A mixture according to claim 1, wherein the mixture self-hardens at body temperature.

4. A mixture according to claim 1, wherein the low weight percent of PVA is between about 1% to about 15%.

5. A mixture according to claim 1, wherein the mixture is viscous and flowable at temperatures of between about 80° F.-85° F.

6. A mixture according to claim 1, wherein the mixture hardens to a flexible solid mass in less than about 60 minutes when exposed to temperatures at about 98.6° F.

7. A mixture according to claim 1, wherein the primary mixture comprises calcium derivatives.

8. A mixture according to claim 1, wherein the bone cement solid mass is a spinal intervertebral bone cement for repairing or treating intevertebral bones.

9. An orthopaedic medical kit, comprising:
a sterile container of flowable bone cement comprising a low weight percent of PVA with a molecular weight between about 124,000 to about 165,000.

10. A kit according to claim 9, wherein the container defines a therapeutic delivery device for placing the flowable bone cement in local target bone structure in a desired quantity.

11. A kit according to claim 9, wherein the container communicates with a therapeutic delivery device for placing the flowable bone cement in local target bone structure in a desired quantity.

12. An orthopaedic medical kit, comprising:
at least one container of primary bone cement material; and
at least one other container of a sterile solution of low weight percent PVA having a molecular weight between about 124,000 to about 165,000.

13. An orthopaedic medical kit, comprising:
a sterile container of a quantity of flowable bone cement; and
a sterile container of PVA, the PVA having a molecular weight between about 124,000 to about 165,000, wherein when mixed with the quantity of flowable bone cement, the PVA forms a low weight percent of the mixture.

14. A preformed bone cement implant comprising a low weight percent of PVA solution, the PVA having a molecular weight of between about 124,000 to about 165,000, and wherein the PVA material is distributed throughout the implant.

15. A bone cement implant according to claim 14, wherein the implant is a dental implant or void filler for a tooth.

16. A method of embedding bone cement at a boney site, comprising:
providing a flowable viscous mixture comprising a primary material and a low weight percent amount of PVA material; and
introducing the flowable viscous mixture to a target bony treatment site whereby the flowable viscous mixture hardens to a solid mass.

17. A method according to claim 16, wherein the providing step comprises mixing contents of a first container of a PVA solution that has the low weight percent of PVA with contents of a second container housing the primary material.

18. A method according to claim 16, wherein the target boney treatment site is an intevertebral disc.

19. A method of fabricating a biocompatible bone cement, comprising:
providing a primary material or mixture of materials defining a flowable bone cement mixture that hardens to a solid mass when placed in situ; and
mixing a low weight percent amount of PVA solution with the flowable bone cement, wherein, when combined with the flowable bone cement mixture, the PVA solution does not substantially change the viscosity of the flowable bone cement mixture and does not substantially change a time to harden the bone cement in situ.

20. A method according to claim 18, wherein the PVA material is not cured nor polymerized.
